(19) United States
(12) Patent Application Publication
(10) Pub. No.: US 2011/0212474 A1
(43) Pub. Date: Sep. 1, 2011

(54) ABNORMAL ALTERATIONS OF PKC ISOZYMES PROCESSING IN ALZHEIMER'S DISEASE PERIPHERAL CELLS

(75) Inventors: Tapan Kumar Khan, Morgantown, WV (US); Daniel L. Alkon, Bethesda, MD (US)

(73) Assignee: BLANCHETTE ROCKEFELLER NEUROSCIENCES INSTITUTE, MORGANTOWN, WV (US)

(21) Appl. No.: 12/895,957

(22) Filed: Oct. 1, 2010

Related U.S. Application Data
(60) Provisional application No. 61/248,361, filed on Oct. 2, 2009.

(51) Int. Cl. C12Q 1/48

(52) U.S. Cl. 435/15

(57) ABSTRACT

The present invention provides a method for the diagnosis of AD from non-AD conditions by using a PKC Isozyme Index obtained by determining ratios of ratios of different PKC Isozymes in peripheral cells of a test subject in the absence and presence of a beta-amyloid peptide, and optionally, in the presence of a PKC activator.

This application claims the benefit of U.S. Provisional Application Ser. No. 61/248,361, filed on Oct. 2, 2009, the disclosure of which is incorporated herein by reference in its entirety.

### FIELD OF THE INVENTION

The present invention relates to methods of diagnosing Alzheimer's Disease or confirming the presence or absence of Alzheimer's Disease in a subject. The present invention also relates to methods of screening for lead compounds that may be used for the development of therapeutic agents useful in treating or preventing Alzheimer's Disease. The invention also relates to methods of diagnosing Alzheimer's Disease in a subject by detecting alterations the processing of certain PKC isozymes using algorithmic ratios constructed from levels of steady-state or phosphorylated PKC isozymes. The method described herein is useful for diagnosing Alzheimer's Disease, monitoring Alzheimer's Disease progression, and in screening methods for the identification of lead compounds. The invention also relates to methods for selecting patients who have increased responsiveness to treatment of Alzheimer's Disease.

### BACKGROUND OF THE INVENTION

The β-amyloid protein (Aβ) is the major constituent of the neuritic plaques that are, together with the neurofibrillar tangles, physiologic hallmarks of Alzheimer's Disease (AD). Katzman, N Eng J. Med. 1986; 314:964-973; Bush et al., Pharmacol Ther. 1992; 56:97-117. Excessive release of Aβ in different cerebral areas, promoted by a mutant form of amyloid precursor protein (APP), contributes to its accumulation within the neuritic plaques. Wallace, Biochim Biophys Acta. 1994; 1227:183-187. In many cell types from AD tissues, including fibroblasts, changes have been demonstrated in signal transduction systems that involve calcium homeostasis, ion channel permeability, cyclic AMP, and phosphoinositide metabolites. Altered production of Aβ also has been shown. Furthermore, Aβ itself can affect the same transduction systems.

Protein kinase C (PKC) is one of the largest gene families of protein kinase. Liu and Heckman, Cellular Signalling. 1998; 10 (8):529-42. Several PKC isozymes are expressed in the brain, including PKC-α, PKC-β1, PKC-βII, PKC-δ, PKC-ε, and PKC-γ. PKC is primarily a cytosolic protein, but with stimulation it translocates to the membrane.

PKC has been shown to be involved in numerous biochemical processes relevant to AD. PKC activation has a crucial role in learning and memory enhancement, and PKC activators have been shown to increase memory and learning. Sun and Alkon, Eur J. Pharmacol. 2005; 512:43-51; Alkon et al., Proc Natl Acad Sci USA. 2005; 102:16432-16437. PKC activation also has been shown to induce synaptogenesis in rat hippocampus, suggesting the potential of PKC-mediated anti-apoptosis and synaptogenesis during conditions of neurodegeneration. Sun and Alkon, Proc Natl Acad Sci USA. 2008; 105 (36): 13620-13625. Postischemic/hypoxic treatment with bryostatin-1, a PKC activator, effectively rescued ischemia-induced deficits in synaptogenesis, neurotrophic activity, and spatial learning and memory. Sun and Alkon, Proc Natl Acad Sci USA. 2008. This effect is accompanied by increases in levels of synaptic proteins spiniophilin and synaptophysin and structural changes in synaptic morphology. Hongpaisan and Alkon, Proc Natl Acad Sci USA. 2007; 104:19571-19576. Bryostatin-induced synaptogenesis for long-term associative memory is also regulated by PKC activation. Hongpaisan and Alkon, PNAS 2007. PKC also activates neurotrophin production. Neurotrophins, particularly brain-derived neurotrophic factor (BDNF) and nerve growth factor (NGF), are key growth factors that initiate repair and regrowth of damaged neurons and synapses. Activation of some PKC isozymes, particularly PKC-ε and PKC-α, protect against neurological injury, most likely by upregulating the production of neurotrophies. Weinreb et al., The FASEB Journal. 2004; 18:1471-1473). PKC activators are also reported to induce expression of tyrosine hydroxylase and induce neuronal survival and neurite outgrowth. Du and Iacovitti, J. Neurochem. 1997; 68: 564-69; Hongpaisan and Alkon, PNAS 2007; Lallemend et al., J. Cell Sci. 2005; 118: 4511-25.

The PKC gene family consists presently of 11 genes which are divided into four subgroups: 1) classical PKC-α, -β1, -β2 (β1 and β2 are alternatively spliced forms of the same gene) and -γ, 2) novel PKC-δ, -ε, -η and -θ; 3) atypical PKC-ξ, -80 , -η and -86 , -λ, -η and -ι; and 4) PKC-μ. PKC-μ resembles the atypical PKC isozymes but differs by having a putative transmembrane domain. Blohe et al., Cancer Metast. Rev. 1994; 13: 411; Ilug et al., Biochem J. 1993; 291:329; Kikkawa et al., Ann. Rev. Biochem. 1989; 58:31. The -α, -β1, -β2, and -γ isozymes are Ca²⁺, phospholipid and diacylglycerol-dependent and represent the classical isoforms of PKC, whereas the other isozymes are activated by phospholipids and diacylglycerol but are not dependent on Ca²⁺. All isozymes encompass five variable (V1-V5) regions, and the α, β, γ isozymes contain four (C1-C4) structural domains which are highly conserved. All isozymes except PKC-α, -β and -γ lack the C2 domain, and the -λ, -η and isozymes also lack nine of two cysteine-rich zinc finger domains in C1, to which diacylglycerol binds. The C1 domain also contains the pseudosubstrate sequence which is highly conserved among all isozymes, and which serves an autoregulatory function by blocking the substrate-binding site to produce an inactive conformation of the enzyme. House et al., Science. 1987; 238: 1726.

Because of these structural features, diverse PKC isozymes are thought to have highly specialized roles in signal transduction in response to physiological stimuli. Responses of various PKC isozymes to stimuli have been studied in AD. For example, AD patients have reduced levels of PKC-α/ε-mediated phosphorylation of Erk1/2, a major downstream substrate of PKC. Khan and Alkon, Proc Natl Acad Sci USA. 2006; 103:13203-13207. In addition, Aβ peptide application to normal fibroblasts reduces PKC activity, because Aβ directly down-regulates PKC α/ε. PKC activators, especially those specific for PKC α/ε, have been proposed to counteract the effect of Aβ, and thereby reverse or prevent the Aβ-induced changes.

PKC has also proven to modulate APP processing. PKC activators have been shown to significantly increase the relative amount of non-amyloidogenic soluble APP (sAPP) secreted by cells. PKC activation also reversed the abnormal MAP kinase phosphorylation and concomitant elevated levels of Aβ in AD fibroblasts. See U.S. Patent Application Publication No. US-2007-0082366. Furthermore, one potent PKC activator, bryostatin, was found to reduce Aβ (1-42) levels in the brains of transgenic mice with human AD genes.

Conversely, Aβ peptides have also been shown to differentially affect PKC isozymes in AD fibroblasts compared with non-AD fibroblasts. Favit et al., Proc. Natl. Acad. Sci. USA; 1998.95: 5562-67. Treatment of non-AD (AC) fibroblasts with nanomolar concentrations of Aβ (1-40) resulted in a 75% decrease in PKC-α, which is already reduced in AD fibroblasts, but not PKC-γ immunoreactivity. In contrast, in AD fibroblasts, Aβ (1-40) caused a 70% reduction of PKC-γ but not PKC-α immunoreactivity. Treatment with a PKC activator restored the PKC-α signal in AC cells but it did not reverse the effects on PKC-γ in the AD cells. Treatment with a protein synthesis inhibitor did not inhibit the effects of Aβ (1-40) in AD cells but did inhibit the effects in AC cells treated with the PKC activator, suggesting that PKC activation exerts a protective role via de novo protein synthesis in normal but not AD cells.

The present invention provides methods for exploiting the effect of Aβ-induced changes on levels of various PKC isozymes in peripheral cells. Measuring levels steady state and/or phosphorylated PKC isozymes, together with Aβ-induced changes, can be used to diagnose AD, monitor the progression of AD or from a non-AD state to AD, and in screening methods to find therapeutics treating AD.

### SUMMARY OF THE INVENTION

The present invention is directed to methods for determining or confirming the presence or absence of Alzheimer's Disease in a subject. In one embodiment, the method comprises i) determining the steady state protein levels of a first PKC isozyme in cells from a candidate subject in the absence of and in the presence of an Aβ peptide to generate a first ratio; ii) determining the steady state protein levels of a second PKC isozyme in the absence of and in the presence of the Aβ peptide, wherein the second PKC isozyme is not known to be modulated by the Aβ peptide, to generate a second ratio; iii) generating a PKC isozyme Index by dividing the first ratio by the second ratio.

In one embodiment, the Aβ peptide is Aβ (1-42), although any Aβ peptide may be used.

In another embodiment, the first PKC isozyme is PKC-α, and/or PKC-ε and the second PKC isozyme is PKC-γ.

In one specific embodiment, differential processing of steady state PKC isozymes levels, the PKC isozyme Index, is determined according to the following equation:

$$\frac{[ PKC \text{-} α ] / {[ PKC \text{-} α ]}_{A β}}{[ PKC \text{-} γ ] / {[ PKC \text{-} γ ]}_{A β}} = PKC \text{-} α Index \qquad ( Eq . 1 )$$

In a further embodiment, the PKC-α Index from the test subject is compared with the PKC-α Index of cells from a non-AD control subject. In a specific embodiment, the cells of the control subject are of the same cell type and are from an age-matched non-AD control subject (AC).

In one embodiment, a PKC-α Index from cells of the test subject that is lower than the PKC-α index from cells of the control subject (AC) is indicative of AD.

In another specific embodiment, the subject is diagnosed with AD if the PKC-α Index value is greater than about 1.0.

In another specific embodiment, differential processing of steady state PKC isozyme levels is determined using an ratio according to the following equation:

$$\frac{[ PKC \text{-} ɛ ] / {[ PKC \text{-} ɛ ]}_{A β}}{[ PKC \text{-} γ ] / {[ PKC \text{-} γ ]}_{A β}} = PKC \text{-} ɛ Index \qquad ( Eq . 2 )$$

In a further embodiment, the PKC-ε Index from the test subject is compared with the PKC-ε Index of cells from a non-AD control subject. In a specific embodiment, the cells of the control subject are of the same cell type and are from an age-matched non-AD control subject (AC).

In one embodiment, a PKC-ε index from cells of the test subject that is lower than the PKC-ε index from cells from the control subject (AC) is indicative of AD.

In another specific embodiment, the subject is diagnosed with AD if the PKC-ε Index value is greater than about 1.0.

In another specific embodiment, the method comprises i) determining protein levels of a first phosphorylated PKC isozyme in cells from a candidate subject in the absence of and in the presence of an Aβ peptide to generate a first ratio; ii) determining the protein levels of a second phosphorylated PKC isozyme in the absence of and in the presence of the Aβ peptide, wherein the second PKC isozyme is not known to be modulated by the Aβ peptide, to generate a second ratio; iii) generating a phosphorylated PKC isozyme Index by dividing the first ratio by the second ratio.

In a specific embodiment, the differential processing of phosphorylated PKC isozymes, the phosphorylated PKC (p-PKC) isozyme Index, is determined according to the following equation:

$$\frac{[ p \text{-} PKC \text{-} α ] / {[ p \text{-} PKC \text{-} α ]}_{A β}}{[ p \text{-} PKC \text{-} γ ] / {[ p \text{-} PKC \text{-} γ ]}_{A β}} = p \text{-} PKC \text{-} α Index \qquad ( Eq . 3 )$$

In a further embodiment, the p-PKC-α Index from the test subject is compared with the p-PKC-α Index of cells from a non-AD control subject. In a specific embodiment, the cells of the control subject are of the same cell type and are from an age-matched non-AD control subject (AC).

In one embodiment, a p-PKC-α index from cells of the test subject that is lower than the p-PKC-α index from cells of the control subject (AC) is indicative of AD.

In another specific embodiment, the subject is diagnosed with AD if the p-PKC-α Index value is greater than about 1.0.

In another specific embodiment, the differential processing of phosphorylated PKC isozymes is determined using an ratio according to the following equation:

$$\frac{[ p \text{-} PKC \text{-} ɛ ] / {[ p \text{-} PKC \text{-} ɛ ]}_{A β}}{[ p \text{-} PKC \text{-} γ ] / {[ p \text{-} PKC \text{-} γ ]}_{A β}} = p \text{-} PKC \text{-} ɛ Index \qquad ( Eq . 4 )$$

In a further embodiment, the p-PKC-ε Index from the test subject is compared with the p-PKC-ε Index of cells from a non-AD control subject. In a specific embodiment, the cells of the control subject are of the same cell type and are from an age-matched non-AD control subject (AC).

In one embodiment, a p-PKC-ε Index from cells of the test subject that is lower than the p-PKC-ε Index from cells from the control subject (AC) is indicative of AD.

In another specific embodiment, the subject is diagnosed with AD if the p-PKC-ε Index value is greater than about 1.0.

In yet a further specific embodiment, subject cells are contacted with a PKC activator at concentrations sufficient to induce phosphorylation the first and/or second PKC isozymes in the absence and in the presence of Aβ, and determining the PKC index according to Equations 1, 2, 3 and/or 4, above.

In a further embodiment, the indices determined by the above equations are compared with those of cells from a non-AD control subject. In a specific embodiment, the cells are of the same cell type and are from an age-matched non-AD control subject (AC).

In another specific embodiment, the present invention provides methods for monitoring the progression from a pre-AD state, such as mild cognitive impairment (MCI), or from an earlier stage of the disease, such as early stage AD, to AD, using the above described methods. In this embodiment, the methods of the present invention are repeated at temporal intervals and a reduction in the PCK Index over time is indicative of progression of non-AD to AD, or progression of AD from early to late stages.

In certain embodiments of the invention, the cells that are used in the diagnostic assays are peripheral cells. In some embodiments, the cells are skin cells, skin fibroblast cells, blood cells or buccal mucosa cells.

In a specific embodiment, the cells are skin fibroblast cells.

In another embodiment, the present invention is provides methods for identifying a lead compound useful for the treatment of AD, comprising: contacting cells isolated from a subject diagnosed with AD with a test compound followed by determining the effect i) on the PKC-α Index according to Equation 1; ii) the effect on the PKC-ε Index according to Equation 2; iii) on the p-PKC-α Index according to Equation 3; and/or iv) on the p-PKC-ε Index according to Equation 4, wherein the PKC index (or combination of indices) increases in the presence of the test compound compared with the same index or indices measured in the absence of the test compound.

In another embodiment, the present invention provides kits containing reagents or instruments useful for the detection or diagnosis of AD. In some embodiment, the kits contain one or more Aβ peptides such as Aβ (1-40) and/or Aβ (1-42); antibodies specific for steady state and phosphorylated PKC isozymes; one or more protein samples of PKC isozymes for use as controls in the immunoassay; and instructions for carrying out the immunoassay and containing criteria for evaluating the results.

In further embodiments, the kits may also contain instruments, buffers and storage containers necessary to perform one or more biopsies, such as punch skin biopsies.

### DESCRIPTION OF THE DRAWINGS

FIG. 1: FIG. 1 depicts a comparison of the PKC-α Index (Equation 1) between AD cells and age-matched control cells (AC).

### DETAILED DESCRIPTION OF THE INVENTION

The present invention relates, in certain aspects, to methods of diagnosing Alzheimer's Disease in human cells taken from subjects that have been identified for testing and diagnosis. The diagnosis is based upon the discovery that differential levels of either steady state or phosphorylated PKC in peripheral cells taken from a subject, together with Aβ-induced changes in same, can be used to construct algorithmic ratios to determine whether a subject has AD.

The method depends on measuring levels of steady state or phosphorylated PKC isozymes in peripheral cells from a candidate subject and, optionally, from a non-AD control subject (AC). Sequentially or concurrently, steady levels of a first PKC isozyme are measured in peripheral cells from the AD and AC subjects both in the absence of, and in the presence of, an Aβ peptide to generate a first ratio of the PKC isozyme level (PKC isozyme level in the absence of Aβ peptide/level in the presence of Aβ peptide). A second PKC isozyme ratio is also obtained by measuring steady state or phosphorylated levels of a second PKC isozyme in peripheral cells from a subject, again in the absence of and in the presence of an Aβ peptide. Results of these measurements are then used to construct a third ratio, in which the first ratio (level of the first PKC isozyme obtained in cells not contacted with the Aβ peptide/level of the first PKC isozyme obtained in cells contacted with the Aβ peptide) is divided by the second ratio (level of the second PKC isozyme in cells not contacted with the Aβ peptide/level of the second PKC isozyme in cells contacted with the Aβ peptide) to generate a PKC Isozyme Index. This PKC Isozyme Index can be generated using the following general equations:

$$I \frac{[ PKC \text{-} x ] / {[ PKC \text{-} x ]}_{A β}}{[ PKC \text{-} z ] / {[ PKC \text{-} z ]}_{A β}} = PKC \text{-} x Index$$

$$II \frac{[ p \text{-} PKC \text{-} x ] / {[ p \text{-} PKC \text{-} x ]}_{A β}}{[ p \text{-} PKC \text{-} z ] / {[ p \text{-} PKC \text{-} z ]}_{A β}} = p \text{-} PKC \text{-} x Index$$

or
where “x” represents a PKC isozyme of interest, “z” represents the second PKC isozyme, “p-PKC-x” and “p-PKC-z” represent phosphorylated PKC isozymes, and Aβ represents the cells in which the levels of the PKC isozymes are determined in the presence of an Aβ peptide.

In some embodiments, PKC-x and p-PKC-x represent a PKC isozyme known to be differentially affected by an Aβ peptide in AD compared with non-AD cells and PKC-z and PKC-z represent a PKC isozyme that is not known to be differentially affected by an Aβ peptide in AD compared with non-AD cells. In particular, it is contemplated that PKC-x is PKC-α or PKC-ε, and PKC-z is PKC-γ.

In one specific embodiment, the invention provides a method for diagnosing the presence or absence of AD by generating a PKC-α Index according to the following Equation 1:

$$\frac{[ PKC \text{-} α ] / {[ PKC \text{-} α ]}_{A β}}{[ PKC \text{-} γ ] / {[ PKC \text{-} γ ]}_{A β}} = PKC \text{-} α Index \qquad ( Eq . 1 )$$

It has been unexpectedly discovered that a PKC-α Index from non-AD subjects, even subjects having non-AD dementia or amnesia, will be higher than the same PKC index from patients having AD.

In one embodiment, if the PKC-α Index value is about 1.0 or less, this is diagnostic of AD.

In another specific embodiment, the invention provides a method for diagnosing the presence or absence of AD by generating a PKC-ε Index according to the following Equation 2:

$$\frac{[ PKC \text{-} ɛ ] / {[ PKC \text{-} ɛ ]}_{A β}}{[ PKC \text{-} γ ] / {[ PKC \text{-} γ ]}_{A β}} = PKC \text{-} ɛ Index \qquad ( Eq . 2 )$$

It has been unexpectedly discovered that a PKC-ε Index from non-AD subjects, even subjects having non-AD dementia or amnesia, will be higher than the same PKC index from patients having AD.

In one embodiment, if the PKC-ε Index value is about 1.0 or less, this is diagnostic of AD.

The methods of the present invention can also be used in conjunction with a PKC activator. In this embodiment, the cells would be contacted with a PKC activator in the absence or presence of Aβ, and the levels of the PKC isozymes according to the above Equations I and II would be used to determine a PKC Index of the present invention.

Protein kinase C activators that are specifically contemplated for use in the diagnostic methods, kits and methods of screening to identify compounds of the instant invention include, but are not limited to: macrocyclic lactone, benzolactam, or pyrrolidinone. bradykinin; bryostatin 1; bryostatin 2-18; neristatin; phorbol esters; bradykinin, bombesin, cholecystokinin, thrombin, prostaglandin F2α and vasopressin. Also included are compounds known as “bryologs,” which are derivatives of bryostatins. While bryostatin-1 has two pyran rings and one 6-membered cyclic acetal, in most bryologs one of the pyrans of bryostatin-1 is replaced with a second 6-membered acetal ring. See PCT WO20081100449. Finally, epoxidized and cyclopropanated polyunsaturated fatty acids have been identified as PKC-ε selective activators. See pending PCT Serial No. PCT US/2009/051927, filed on Jul. 28, 2009.

According to the present invention, the term “PKC isozyme” refers to the -α, -β1, -β2 -γ, -δ, -ε, -η, -θ, -86 , -80 , -η, -ι, and -μ isozymes. In a specific embodiment, the term PKC isozyme refers to the -α, -β, -γ, -δ, and -ε.

The term “Aβ peptide” refers to a peptide of 39-43 amino acids length from the membrane protein, Amyloid Precursor Protein (APP), that appears to be the main constituent of amyloid plaques in the brains of Alzheimer's Disease patients. In specific embodiments, the Aβ peptide used in the methods of the present invention is Aβ (1-40) and/or Aβ (1-42). The terms “amyloid beta peptide”, “beta amyloid protein”, “beta amyloid peptide”, “beta amyloid”, are used interchangeably. Multiple isoforms of APP exist, for example APP⁶⁹⁵, APP⁷⁵¹, and APP⁷⁷⁰. Examples of specific isotypes of APP which are currently known to exist in humans are the 695 amino acid polypeptide described by Kang et. al., Nature. 1987; 325:733-736 which is designated as the “normal” APP; a 751 amino acid polypeptide described by Ponte et al., Nature. 1988; 331:525-527 and Tanzi et al., Nature. 1988; 331:528-530; and a 770-amino acid polypeptide described by Kitaguchi et. al. Nature. 1988; 331:530-532. As a result of proteolytic processing of APP by different α- and β-secretase enzymes in vivo or in situ, Aβ is found in both a “short form,” 40 amino acids in length, and a “long form,” ranging from 42-43 amino acids in length.

Aβ peptides are commercially available, e.g., from rPeptides (Bogart, Ga.) or GenScript (Piscataway, N.J.). In addition, Aβ peptides can be synthesized or generated using recombinant engineering techniques according to known methods.

The present invention also provides methods for monitoring methods of monitoring the progression of AD in a subject. As AD progresses, such as from early AD or mild AD, to moderate AD or to advanced AD, the PKC Isozyme Index value, determined as described above, is expected to decrease, or even become negative, compared with the PKC Isozyme Index value from an early stage of AD, or a PKC Isozyme Index Value from a pre-AD condition.

As used herein, “early stage Alzheimer's Disease” means the stage of the disease. Persons with early-stage AD and related dementias have only mild impairment due to the symptoms of the disease. They may still be working, driving and need only minimal assistance with certain activities of daily living. Individuals in this stage are often self-aware of their diagnosis and abilities.

“Mild Alzheimer's Disease” refers to a stage where cognitive decline is more evident. A subject with mild AD may be forgetful of recent events or personal details. Other problems include impaired mathematical ability (for instance, difficulty counting backwards from 100 by 9 s), a diminished ability to carry out complex tasks like throwing a party or managing finances, moodiness, and social withdrawal.

As used here in, “non-AD dementia” refers to conditions that share symptoms with AD. These conditions include mild cognitive impairment, vascular dementia such as that caused by stroke or head trauma, mixed dementia, dementia with Lewy Bodies, Parkinson's Disease, Frontotemporal dementia, Creutzfeldt-Jakob Disease or another infectious disease, Pick's Disease, Huntington's Disease, Wernicke-Korsakoff Syndrome.

“Mild Cognitive Impairment” refers to a condition characterized by memory problems greater than normally expected with aging, but does not show other symptoms of dementia, such as impaired judgment or reasoning. Ten to 15 percent of people with MCI develop AD every year compared to one percent of the normal elderly population. “Amnestic MCI” is a type of MCI that involves short-term memory loss.

A “non-AD control subject” according to the present invention refers to a subject who has not been diagnosed with or suspected of having AD. Such a subject can include a subject having non-AD dementia or amnesia.

In the methods of the invention, the peripheral cells that are taken from the individual or patient can be any viable cells. In one embodiment, the cells are skin fibroblasts, but any other peripheral tissue cell (i.e. tissue cells outside of the central nervous system) may be used in the tests of this invention if such cells are more convenient to obtain or process. Other suitable cells include, but are not limited to, blood cells such as erythrocytes and lymphocytes, buccal mucosal cells, nerve cells such as olfactory neurons, cerebrospinal fluid, urine and any other peripheral cell type. In addition, the cells used for purposes of comparison do not necessarily have to be from healthy donors.

The cells may be fresh or may be cultured (see, U.S. Pat. No. 6,107,050, which is herein incorporated by reference in its entirety). In a specific embodiment, a punch skin biopsy can be used to obtain skin fibroblasts from a subject. These fibroblasts are analyzed directly using the techniques described herein or introduced into cell culture conditions. The resulting cultured fibroblasts are then analyzed as described in the examples and throughout the specification. Other steps may be required to prepare other types of cells which might be used for analysis such as buccal mucosal cells, nerve cells such as olfactory cells, blood cells such as erythrocytes and lymphocytes, etc. For example, blood cells can be easily obtained by drawing blood from peripheral veins. Cells can then be separated by standard procedures (e.g. using a cell sorter, centrifugation, etc.) and later analyzed.

According to the methods of the present invention, the concentration of Aβ peptide used can be from about 1 nM to 100 μM, preferably from about 10 nM to 10 μM. Cells should be between about 80-100% confluent when treated.

Proteins may be isolated from the cells by conventional methods known to one of skill in the art. In a preferred method, cells isolated from a patient are washed and pelleted in phosphate buffered saline (PBS). Pellets are then washed with “homogenization buffer” comprising 50 nM NaF, 1 mM EDTA, 1 mM EGTA, 20 μg/ml leupeptin, 50 μg/ml pepstatin, 10 mM TRIS-HCl, pH=7.4, and pelleted by centrifugation. The supernatant is discarded, and “homogenization buffer” is added to the pellet followed by sonication of the pellet. The protein extract may be used fresh or stored at −80° C. for later analysis.

In the methods of the invention, the antibodies used in the disclosed immunoassays may be monoclonal or polyclonal in origin. The phosphorylated and non-phosphorylated PKC isozyme, protein or portions thereof, used to generate the antibodies may be from natural or recombinant sources or generated by chemical synthesis.

In certain embodiments of the diagnostic methods of the invention, PKC isozyme proteins are detected by immunoassay. In certain embodiments of the invention, the immunoassay may be a radioimmunoassay, a Western blot assay, an immunofluoresence assay, an enzyme linked immunosorbent assay (ELISA), an immunoprecipitation assay, a chemiluminescence assay, an immunohistochemical assay, an immunoelectrophoresis assay, a dot blot assay, or a slot blot assay. In further preferred embodiments of the diagnostic methods of the invention, protein arrays or peptide arrays or protein micro-arrays may be employed in the diagnostic methods. Quantitation of protein can be evaluated using e.g., densitometry or spectrophotometry.

In addition, the methods disclosed herein can be used in combination with other diagnostic methods, such as those described in the application based on U.S. Provisional Application Ser. Nos. 61/248,368, 61/344,045, 61/362,518, and 61/365,545, for Fibroblast Growth Patterns for Diagnosis of Alzheimer's Disease, filed Oct. 2, 2009, entitled “Fibroblast Growth Patterns for the Diagnosis of Alzheimer's Disease.” Other methods contemplated for use in combination with the present method are described in U.S. Pat. No. 7,682,807 to Alkon et al., and PCT application nos. PCT/US2004/038160 and PCT/US2005/036014.

The invention is also directed, in certain embodiments, to kits containing reagents or instruments useful for the detection or diagnosis of AD. For example, the kits would contain one or more Aβ peptides such as Aβ (1-40) and/or Aβ (1-42); antibodies specific for steady state and phosphorylated PKC isozymes; one or more protein samples of PKC isozymes for use as controls in the immunoassay; and instructions for carrying out the immunoassay and containing criteria for evaluating the results. The kits may also contain any one or more of the protein kinase C activators disclosed herein (such as, for example, bradykinin or bryostatin). The kits may contain instruments, buffers and storage containers necessary to perform one or more biopsies, such as punch skin biopsies. The kits may also include buffers, secondary antibodies, control cells, and the like.

In further aspects, the invention is directed to methods for screening to identify lead compounds useful for treating AD as well as to methods of using these compounds or chemical derivatives of the lead compounds in pharmaceutical formulations to treat or prevent AD in subjects in need thereof. One such method of screening to identify therapeutic substances would involve the steps of contacting sample cells from an AD patient with a substance being screened herein and then determining the PKC Index. An agent that reverses or improves the AD PKC Index value back to levels found in or non-AD control cells would be identified and selected as a substance potentially useful for the treatment or prevention of AD.

As used herein, “lead compounds” are compounds identified using the methods of screening compounds disclosed herein. Lead compounds may have activity in shifting the Alzheimer's Disease-specific molecular biomarkers disclosed herein, i.e., the PKC Index, to values corresponding to those values calculated for non-Alzheimer's Disease-cells in the assays described herein. Lead compounds may be subsequently chemically modified to optimize or enhance their activity for use in pharmaceutical compositions for the treatment or prevention of Alzheimer's Disease.

Because direct access to neurons in the brains of living human beings is impossible, early diagnosis of Alzheimer's Disease is extremely difficult. By measuring the Alzheimer's Disease-specific molecular biomarkers disclosed herein, the present invention provides highly practical, highly specific and highly selective tests for early diagnosis of Alzheimer's Disease. In addition, the Alzheimer's Disease-specific PKC Index described herein provide a basis for following disease progression and for identifying candidate therapeutic agents for drug development targeted to the treatment and prevention of Alzheimer's Disease.

A great advantage of the instant invention is that the tissue used in the assays and methods disclosed herein may be obtained from subjects using minimally invasive procedures, i.e., without the use of a spinal tap.

### EXAMPLES

### Ex. 1

### Abnormal PKC Isozyme Processing in Alzheimer's Disease Peripheral Cells

Rationale: PKC signaling pathways regulate important molecular events in learning and memory and neurodegenerative pathophysiology of Alzheimer's disease (AD). The causal roles of PKC isozymes have implicated to be deficit in postmortem brains, skin fibroblasts and blood samples of AD patients. PKC-α and PKC-ε directly or indirectly through phosphorylation of Erk regulate all major pathways that are responsible for post-translational processing of α, β and γ secretases, which control the production of Aβ. The effects Aβ treatment on PKC-α and PKC-ε isozyme are more severe compare to PKC-γ. Several diagnostic methods have been examined with PKC-γ as an internal standard. This invention relates to methods of diagnosing AD from age-matched control (AC) cases and other non-AD dementia cases using peripheral tissue. These methods can be use for screening for compounds for the treatment or prevention of AD.

Cell Samples. Samples used in the method of the present invention were as follows:

(1) 10 AD, 10 AC, 10 non-AD dementia

(2) 90% confluent Skin fibroblast cells;

(3) Treatment: 24 hrs., 1 μM Aβ (1-42).

Skin fibroblasts were taken from two different sources: (A) freshly-obtained skin fibroblasts Fresh skin fibroblasts were obtained from a registry with BRNI affiliated organizations and the Johns Hopkins University and its affiliated centers, and (B) banked human skin fibroblasts purchased from the Coriell Institute for Medical Research (Camden, N.J.). The collection and culture of fibroblasts from freshly obtained skin tissue were performed as follows: punch-biopsy skin tissue samples from AD, non-AD dementia patients, and age-match controls were obtained by qualified personnel. Briefly, the outer keratinous layer of the skin tissue (biopsy sample) was removed after thorough rinsing with cold saline solution. The remaining part of the tissue was minced into small pieces (˜1 mm). The pieces were kept in T-25 (25 sq. cm) cell culture flasks. A few hours were allowed for the cells to adhere to the surface of the culture flasks. Three mL of DMEM culture solution containing 45% fetal bovine serum (FBS) and penicillin/streptomycin was carefully added into the flask and placed in a 5% CO₂ and 37° C. incubator for 3 days. After 3 days, 5 mL of additional culture media were added. All flasks were regularly examined and after 7-10 days they became confluent. Cells were trypsinized and expanded according to their number. The total number of cell passages was not allowed to exceed 16. Banked fibroblasts from AD patients and age match controls were maintained and cultured in T25/T75 culture flasks with DMEM culture medium containing 10% fetal bovine serum (FBS). The total number of cell passages was not allowed to exceed 16.

Aβ Peptide Treatment. Fibroblast cell lines from AD and control patients were treated with 1.0 μM Aβ (1-42)(American Peptide. Company, Sunnyvale, Calif.) in DMEM culture medium with 10% fetal bovine serum, for 24 hours in 5% CO₂ and 37° C. incubator after reaching 90-100% confluence. After the 24 hours of incubation with 1.0 μM Aβ (1-42), the medium was removed and washed three times with regular culture medium without serum and kept for 16 hours.

Detection of PKC Isozymes: PKC isozymes described in the assays below were detected by Western blot (immunoblot).

Assay 1: PKC-α, PKC-γ and PKC-ε

Assay 2: p-PKC-α, p-PKC-γ and p-PKC-ε

Assay 3: PKC translocation

Protein extraction was performed as described previously (Favit et al., PNAS, 1998, supra). Briefly, pellets were re-suspended in homogenizing buffer containing 0.1 M HEPES, 0.04 M EDTA, 0.8 M sucrose, 0.01 M phenylmethylsulfonyl fluoride (PMSF), 2.4 units/ml aprotinin, and 1% SDS, and sonicated (ultrasonic homogenizer, Cole-Parmer). Protein concentration was determined according to routine methods. The crude extracts were placed at 4° C. right before immunoblotting analysis was performed.

For Western blot analysis, SDS/PAGE was carried out in a 10% acrylamide gradient gel of 1.5-mm thickness (Invitrogen, San Diego). The crude homogenate was balanced with sample buffer containing 0.5 M TrisHCl (pH 6.8), 10% glycerol, 2% SDS, and 0.5% 2-mercaptoethanol, to a final volume of 20 ml with a total protein concentration of 10 μg/ml. The samples were electrophoresed and transferred overnight into a nitrocellulose paper (Invitrogen). The nitrocellulose was blocked in 1% BSA/95% TBS for 1 h and then incubated with different PKC isozyme monoclonal antibodies (PKC-α, PKC-γ, and PKC-ε; Transduction Laboratories, Lexington, Ky.) for 1 h. Blots were then incubated with an anti-mouse alkaline phosphatase-conjugated antibody (Sigma) for 1 h. Finally, the nitrocellulose was stained with a solution containing 0.1 M TrisHCl (pH 9.6), 0.001 M MgCl, 1% nitroblue tetrazolium (Pierce), and 1% 5-bromo-4-chloro-3-indolyl phosphate toluidine salt (Pierce). All reactions were carried out at room temperature. Immunoblots were digitized on a flatbed scanner and analyzed by quantitative analysis as follows:

Assay 1: Total PKC

$$\frac{[ PKC \text{-} α ] / {[ PKC \text{-} α ]}_{A β}}{[ PKC \text{-} γ ] / {[ PKC \text{-} γ ]}_{A β}} = PKC \text{-} α Index \qquad ( Eq . 1 )$$

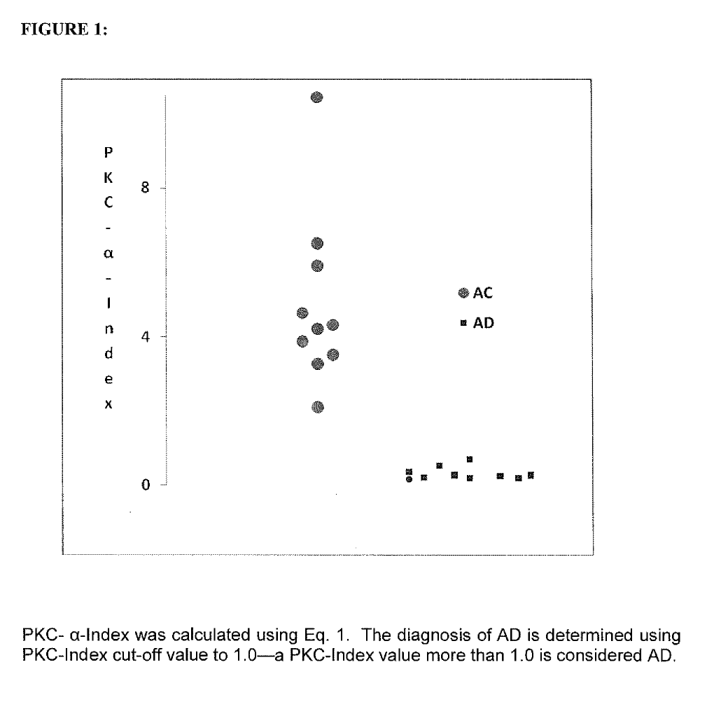

Results of this assay are presented in FIG. 1. It can be seen that the PKC-a indices in the cells AD patients are significantly lower than the PKC-a index taken from the non-AD control subject.

$$\frac{[ PKC \text{-} ɛ ] / {[ PKC \text{-} ɛ ]}_{A β}}{[ PKC \text{-} γ ] / {[ PKC \text{-} γ ]}_{A β}} = PKC \text{-} ɛ Index \qquad ( Eq . 2 )$$

Assay 2: Phospho-PKC

$$\begin{matrix}\frac{[ p \text{-} PKC \text{-} α ] / {[ p \text{-} PKC \text{-} α ]}_{A β}}{[ p \text{-} PKC \text{-} γ ] / {[ p \text{-} PKC \text{-} α ]}_{A β}} = p \text{-} PKC \text{-} α Index & ( Eq . 3 ) \\ \frac{[ p \text{-} PKC \text{-} ɛ ] / {[ p \text{-} PKC \text{-} ɛ ]}_{A β}}{[ p \text{-} PKC \text{-} γ ] / {[ p \text{-} PKC \text{-} γ ]}_{A β}} = p \text{-} PKC \text{-} ɛ Index & ( Eq . 4 )\end{matrix}$$

Patents, patent applications, publications, product descriptions, and protocols are cited throughout this application, the disclosures of which are incorporated herein by reference in their entireties for all purposes.

What is claimed:

1. A method for determining the presence or absence of Alzheimer's Disease in a candidate subject, which method comprises:
i) determining the protein levels of a first PKC isozyme in peripheral cells from a candidate subject in the absence of and in the presence of an Aβ peptide to generate a first ratio;
ii) determining the protein levels of a second PKC isozyme in peripheral cells from a candidate subject in the absence of and in the presence of the Aβ peptide, wherein the second PKC isozyme is not known to be differentially modulated by the Aβ peptide in AD cells compared to non-AD cells, to generate a second ratio;
iii) generating a PKC isozyme Index by dividing the first ratio by the second ratio, wherein a PKC isozyme Index of about 1.0 or lower indicates a diagnosis of Alzheimer's Disease and a PKC isozyme Index of greater than 1.0 indicates the absence of Alzheimer's Disease.

2. The method of claim 1, wherein the PKC isozyme Index is generated using steady state levels of the PKC isozymes as represented by the following Equation I:
$$I \frac{[ PKC \text{-} x ] / {[ PKC \text{-} x ]}_{A β}}{[ PKC \text{-} z ] / {[ PKC \text{-} z ]}_{A β}} = PKC \text{-} x Index$$
wherein “x” represents the first PKC isozyme, “z” represents the second PKC isozyme, and Aβ represents the cells contacted with the Aβ peptide.

3. The method of claim 1, wherein the PKC isozyme Index is generated using phosphorylated levels of the PKC isozymes as represented by the following Equation II:
$$II \frac{[ p \text{-} PKC \text{-} x ] / {[ p \text{-} PKC \text{-} x ]}_{A β}}{[ p \text{-} PKC \text{-} z ] / {[ p \text{-} PKC \text{-} z ]}_{A β}} = p \text{-} PKC \text{-} x Index$$
wherein “x” represents the first PKC isozyme, “z” represents the second PKC isozyme, and Aβ represents the cells contacted with the Aβ peptide, and p-PKC-x and p-PKC-z represent phosphorylated PKC isozymes.

4. The method of claim 1, wherein the method further comprises determining the protein levels of the first and second PKC isozymes in steps i and ii in the presence of a PKC activator.

5. The method of claim 1, wherein the first PKC isozyme is PKC-α and the second PKC isozyme is PKC-γ.

6. The method of claim 1, wherein the first PKC isozyme is PKC-ε and the second PKC isozyme is PKC-γ.

7. The method of claim 2, wherein the first PKC isozyme is PKC-α and the second PKC isozyme is PKC-γ.

8. The method of claim 2, wherein the first PKC isozyme is PKC-ε and the second PKC isozyme is PKC-γ.

9. The method of claim 3, wherein the first PKC isozyme is PKC-α and the second PKC isozyme is PKC-γ.

10. The method of claim 3, wherein the first PKC isozyme is PKC-ε and the second PKC isozyme is PKC-γ.

11. The method of claim 1, wherein the Aβ peptide is Aβ (1-40) or Aβ (1-42).

12. The method of claim 1, wherein the peripheral cells are skin cells, skin fibroblast cells, blood cells or buccal mucosa cells.

13. The method of claim 12, wherein the peripheral cells are skin fibroblast cells.

14. The method of claim 1, wherein the Aβ peptide is present at a concentration of from about 1.0 nM to 10 μM.

15. The method of claim 14, wherein the Aβ peptide is present at a concentration of about 1.0 μM

16. A method for monitoring the progression of Alzheimer's Disease in a subject, which method comprises:
i) generating a PKC isozyme Index from peripheral cells of a test subject at a first time point, according to the method of claim 1, wherein the test subject has been diagnosed with Alzheimer's Disease, to obtain a reference PKC isozyme Index for the subject;
ii) generating the same PKC isozyme Index from peripheral cells of the same subject at one or more time points after the first time point;
iii) determining whether there is a decrease in the PKC isozyme Index obtained from the from the one or more time points after the first time point when compared with the PKC isozyme Index from the first time point;
wherein a decrease in the PKC isozyme Index from the one or more time points after the first time point when compared with the PKC isozyme Index from the first time point indicates progression of Alzheimer's Disease.

17. The method of claim 16, wherein the test subject has been diagnosed with early Alzheimer's Disease at the first time point.

18. The method of claim 16, wherein the test subject has been diagnosed with mild Alzheimer's Disease at the first time point.

19. A method for monitoring the progression from a non-Alzheimer's disease condition to Alzheimer's Disease in a subject, which method comprises:
i) generating a PKC isozyme Index from peripheral cells of a test subject at a first time point, according to the method of claim 1, wherein the test subject does not have a diagnosis of Alzheimer's Disease, to obtain a reference PKC isozyme Index for the subject;
ii) generating the same PKC isozyme Index from peripheral cells of the same subject at one or more time points after the first time point;
iii) determining whether there is a decrease in the PKC isozyme Index obtained from the from the one or more time points after the first time point when compared with the PKC isozyme Index from the first time point;
wherein a decrease in the PKC isozyme Index from the one or more time points after the first time point when compared with the PKC isozyme Index from the first time point indicates progression to Alzheimer's Disease.

20. The method of claim 19, wherein the non-Alzheimer's disease condition is mild cognitive impairment.

21. The method of claim 20, wherein the mild cognitive impairment is amnestic cognitive impairment.

22. A kit comprising one or more Aβ peptides, at least one antibody specific for a PKC isozyme known to be differentially modulated by the Aβ peptide in AD cells compared to non-AD cells; at least one antibody specific for a PKC isozyme that is not known to be differentially modulated by the Aβ peptide in AD cells compared to non-AD cells; and instructions for determining a PKC Isozyme Index.

23. The kit of claim 22, wherein the Aβ peptide is Aβ (1-40) or Aβ (1-42).

24. The kit of claim 22, wherein the PKC isozyme known to be differentially modulated by the Aβ peptide in AD cells is PKC-α.

25. The kit of claim 22, wherein the PKC isozyme known to be differentially modulated by the Aβ peptide in AD cells is PKC-ε.

26. The kit of claim 22, wherein the PKC isozyme known not to be differentially modulated by the Aβ peptide in AD cells is PKC-γ.
